(19) United States
(12) Patent Application Publication
(10) Pub. No.: US 2011/0066078 A1
(43) Pub. Date: Mar. 17, 2011

(54) PAIN MONITOR FOR A PATIENT UNDERGOING A MEDICAL PROCEDURE

(75) Inventors: Armen P. Sarvazyan, Lambertville, NJ (US); Sergey Tsyuryupa, Westampton, PA (US)

(73) Assignee: ARTANN LABORATORIES, INC., Lambertville, NJ (US)

(21) Appl. No.: 12/558,775

(22) Filed: Sep. 14, 2009

(51) Int. Cl. A61B 5/00

(52) U.S. Cl. 600/587

(57) ABSTRACT

A pain monitor for a patient undergoing a medical procedure includes a housing adapted to fit in a hand of the patient, a force sensor, and a battery-powered data processor. The patient is instructed to squeeze the housing with a force representing his level of pain. That force is measured by the sensor, acquired by the data processor and then wirelessly transmitted to an outside data receiver for further processing and recording. This device allows the patient to non-verbally express his perception of the level of pain which in turn allows a physician to adjust the course of the procedure or the level of pain-reducing medication. The pain monitor of the invention is particularly useful during a colonoscopy procedure.

### REFERENCE TO GOVERNMENT-SPONSORED RESEARCH

This invention was made with the U.S. government support under SBIR grant No. R44 DK068936-02 entitled “Colonoscope Force Monitor” and awarded by the National Institute of Health, National Institute of Diabetes and Digestive and Kidney Diseases. The government has certain rights in this invention.

### BACKGROUND OF THE INVENTION

The present invention relates generally to devices useful for monitoring and recording patient's pain and discomfort during various medical procedures. In particular, the invention describes a hand-held device allowing a patient to progressively express the level of pain and discomfort by squeezing the device in his or her hand. Degree of such squeeze is measured and converted to an electronic signal. The magnitude of the signal indicates the degree of pain or discomfort.

The invention is particularly useful for medical procedures when the patient is not sedated or sedated locally or partially. Frequently, the medical practitioner needs to have patient's feedback as to his or her pain in response to certain steps during the procedure.

Examples of such medical procedures include dental procedures, interventional cardiology procedure, and gastro-intestinal evaluation procedures, mainly colonoscopy. At the present time, patients are asked to express their level of discomfort verbally, which is not adequate for objective assessment of patient's pain. Language barriers and difficulties that some patients have deciding on a proper expression of pain assessment (such as with speech-deficient patients) can cause delayed or incorrect perception of the level of patient's discomfort by the medical practitioner. Accurate knowledge of the level of pain would allow the medical practitioner to adjust the course of procedure as well as to titrate properly the amount of anesthetic given to a patient. Limiting anesthetic use to the proper minimum amount would reduce its long-term effect and in some cases may reduce its side effects.

Monitors for assessment of the depth of anesthesia are well known in the art. They are based on collecting a number of vital signs including ECG and EEG and interpreting them to determine if the patient needs more or less anesthetic. One example of such system is shown in the U.S. Pat. No. 7,089,927 by John et al. Another example is found in U.S. Pat. No. 6,826,426 by Lange et al. These devices however are complex, require installation of electrodes, and are generally adapted for completely sedated patients such as those undergoing an open heart surgery.

Hand-held devices for adjusting the infusion rate of pain-reducing medications are also known. These devices incorporate a hand-held button which the patient is instructed to push in order to increase the rate of drug infusion when excessive pain is no longer tolerable. These devices however do not allow the patient to progressively express the degree of pain but rather constitute an ON or OFF pain indicator.

Known also are devices designed to aid the patient in expressing the type of pain and its severity. One such device is described in the U.S. Pat. No. 5,692,500 by Gaston-Johansson. This device includes multiple sets of selection indicators and sliding scales, each being indicative of variations of a respective dimension of pain selected from the group consisting of nature, intensity, location, duration, continuity and pain relief. The selection indicators are mounted on the tool in association with pain descriptors which are designed to help the patient in deciding on the proper way to report pain. This device still fails to address the needs for pain reporting during certain medical procedures like colonoscopy. A similar device is also described in the U.S. Pat. No. 5,018,526 by the same inventor.

A simplified version of a pain indicator is found in the U.S. Pat. No. D493,537 by Abric. It is essentially a ruler with a slider. The patient is asked to move the slider to an appropriate position indicating his level of discomfort. This simple device is not adapted however to generate an electronic signal which can be transmitted to a data acquisition system for integration with other parameters collected and recorded during the medical procedure.

The preferred area of interest for the device of the present invention is in medicine, and more particularly in colonoscopy. Colonoscopy is the preferred method to screen for colorectal cancer, a disease that affects 150 thousand patients per year in the US. Several million screening, diagnostic and therapeutic colonoscopies are performed each year in the U.S. hospitals and ambulatory surgery centers. Colonoscopy requires a physician to inspect the colonic mucosal surface by applying force to a colonoscope and advancing this flexible tube through a series of stationary and movable colonic loops. Pain indication data available to a physician in real time would allow for safer and less painful procedures.

The need therefore exists for a pain monitor allowing a patient to non-verbally express progressively increasing levels of pain and discomfort during a medical procedure, such monitor capable of generating an electronic signal indicative of the level of patient's pain.

### SUMMARY OF THE INVENTION

Accordingly, it is an object of the present invention to overcome these and other drawbacks of the prior art by providing a novel pain monitor allowing a patient to non-verbally express various levels of pain and discomfort during a medical procedure.

It is another object of the invention to provide a pain monitor capable of generating an electronic signal corresponding to various levels of pain expressed by a patient.

It is a further object of the invention to provide a pain monitor capable of providing pain level data in real time throughout the duration of the medical procedure.

It is yet a further object of the invention to provide a pain monitor allowing data transmission to a central data receiver for integrating pain data with other data which is electronically collected during the medical procedure.

The design of the pain monitor of the invention takes advantage of an instinctive motion to clench one's fist when in pain. The pain monitor is placed in the patient's hand and measures the degree of force exerted to clench the fist. This design has two important advantages. First, there is only a minimal training required for a patient to use the device as it is only natural to clench a fist and squeeze the device when in pain. Second, this approach allows using the device even for patients in advanced stages of anesthesia as they tend to clench their fists naturally and often subconsciously.

The pain monitor of the invention is a handheld device which consists of the following main functional units:
a housing shaped and sized to fit in a human hand and optionally equipped with closure means to attach to the palm of the human hand. The patient is instructed to squeeze the housing with a force indicative of the degree of pain he or she experiences;
a sensor to measure the level of squeezing force;
a data processor adapted to convert the force data into a signal indicating the level of pain by the patient and transmit it (via cable or wirelessly) to the data receiver for further processing and recording;
a power source such as a battery to provide electrical power to the sensor and data processing means.

For convenience and simplicity it is preferred to put the electronic components of the device entirely inside the housing and to arrange for data transmission via a wireless protocol such as Bluetooth, Zigbee or others. This however is not essential to the function of the device. Various embodiments are envisioned where at least some of the components are located partially or entirely outside of the housing and data is transmitted via a cable.

### BRIEF DESCRIPTION OF THE DRAWINGS

A more complete appreciation of the subject matter of the present invention and the various advantages thereof can be realized by reference to the following detailed description in which reference is made to the accompanying drawings in which:

FIG. 1 is a general view of the patient pain monitor apparatus according to the first embodiment of the invention,

FIG. 2 is an internal view of the same according to the first embodiment of the invention,

FIG. 3 is a cross-sectional view of the first embodiment of the invention,

FIGS. 4 and 5 show the pain monitor in use by a patient,

FIG. 6 shows an alternative design of the pain monitor according to the second embodiment of the invention, and

FIGS. 7 and 8 show yet another alternative design of the pain monitor according to the third embodiment of the invention.

### DETAILED DESCRIPTION OF THE PREFERRED EMBODIMENTS OF THE INVENTION

A detailed description of the present invention follows with reference to accompanying drawings in which like elements are indicated by like reference letters and numerals.

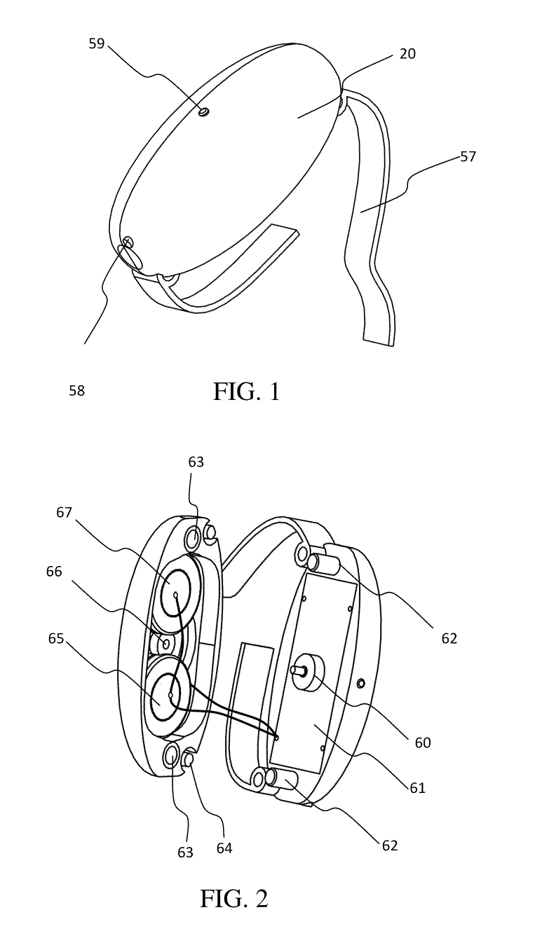

A first preferred embodiment of the pain monitor of the invention is shown in detail in FIGS. 1 through 3. FIG. 1 displays a general view of the patient pain monitor 20 placed on a patient's hand and retained with a strap 57 including a Velcro® or a buckle closure means. Pain monitor 20 is therefore adapted for patient's fingers to continuously grip the device. When experiencing pain, the patient is instructed to squeeze the pain monitor 20 proportionally to the level of pain. Button 58 turns the device on and off. Light indicator 59 displays information about the connection status with the data receiving unit (not shown). It is also envisioned that the patient, who is typically at least partially sedated, will instinctively squeeze the pain monitor when experiencing pain.

The size and shape of the housing of the device is adapted to fit inside a human hand. Several sizes of the housing may be needed to accommodate a range of patients from children through large adults.

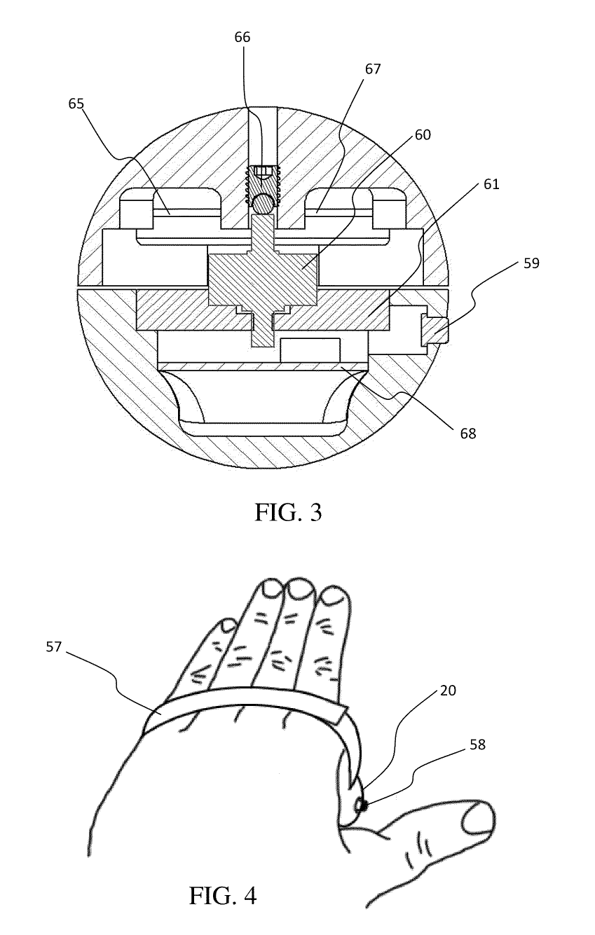

FIG. 2 illustrates the pain monitor 20 having its two rigid halves open so that the internal components can be seen. A force sensor 60 is supported by the holder 61 in one half of the pain monitor 20. Two guiding pins 62 are fixedly placed in one half of the monitor 20 and are adapted to slide within the corresponding slots 63 of the other half of the monitor. They allow for a linear sliding motion of one half towards the other when the pain monitor 20 is squeezed by the patient. The retaining end of the strap 57 is attached to the monitor 20 by the holder 64. Batteries 65 and 67 provide electrical power to the force sensor 60 and the electronic data processor board 68. The board 68 is adapted for signal acquisition, processing and transmission as shown in FIG. 3. It comprises a load cell, an analog-to-digital converter, a microprocessor and a Bluetooth module. The load cell is based on a strain gauge or other force measuring technology. The force sensor with the load cell converts an input mechanical force into an electrical output signal. Analog-to-digital converter provides for data acquisition while the microprocessor provides for data processing, and the Bluetooth module provides for data transfer. The set screw 66 with an optional rotating ball at the end aimed to ensure proper axial alignment is so positioned as to place the other half of the monitor 20 in contact with the free end of the force sensor 60, see FIG. 3.

FIG. 3 also shows a general cross-sectional view of the pain monitor 20. The electronic board 68 is preferably adapted to wirelessly transmit the measurements from the force sensor 60 to the outside data receiver. The light indicator 59 and batteries 65 and 67 can also been seen. The force sensor 60 is sandwiched between both halves of the monitor by the holder 61 on one side and the set screw 66 on the other. Upon squeezing both halves of the pain monitor 20 together, a compression force is applied onto the force sensor 60. This force is measured and transmitted outside as an indicator of patient's pain. Even if the patient is sedated or totally unconscious, the pain monitor is retained in the patient's hand and involuntary clenching of the fist by the patient would still allow recording of the pain level caused during the medical procedure.

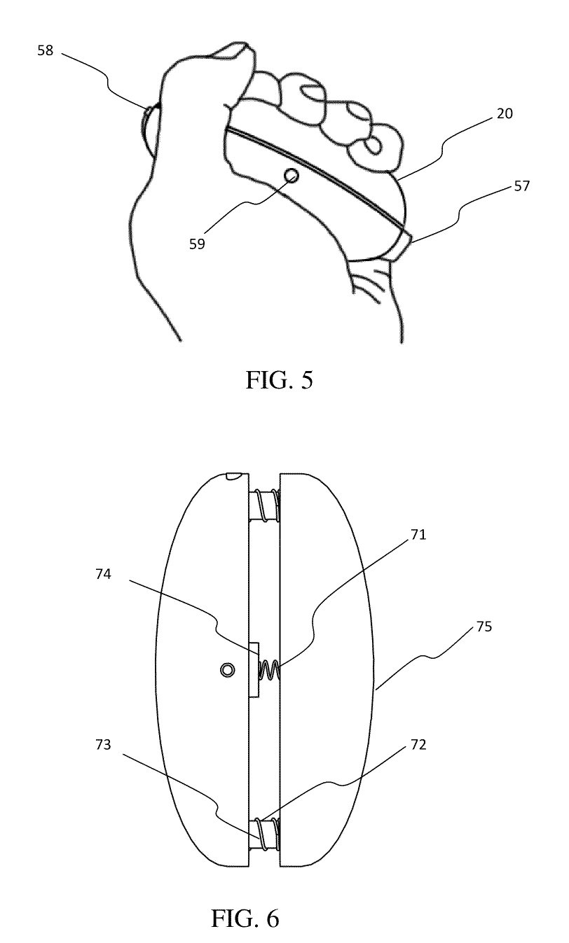

FIG. 4 shows a process of putting a pain monitor 20 over a patient's hand. A Velcro® closure strap 57 allows for adjustment in fitting the device over the patient's palm and holds the pain monitor in the right position to be squeezed by a patient as shown in FIG. 5.

FIG. 6 shows an alternative design of the pain monitor 75 according to the second embodiment of the invention. This design allows the patient to have a better feel for applied force by controlling displacement of one half of the device towards the other. This design is similar to the pain monitor 20 of the first embodiment. The main difference is the presence of a gap between its spaced apart halves. The guiding pins 72 are equipped with springs 73. The load cell 74 monitors the squeeze through measuring the force exerted by the spring 71. Reduction in gap when the halves are squeezed by a patient serves as a biofeedback to the patient as to the degree of squeeze of the device.

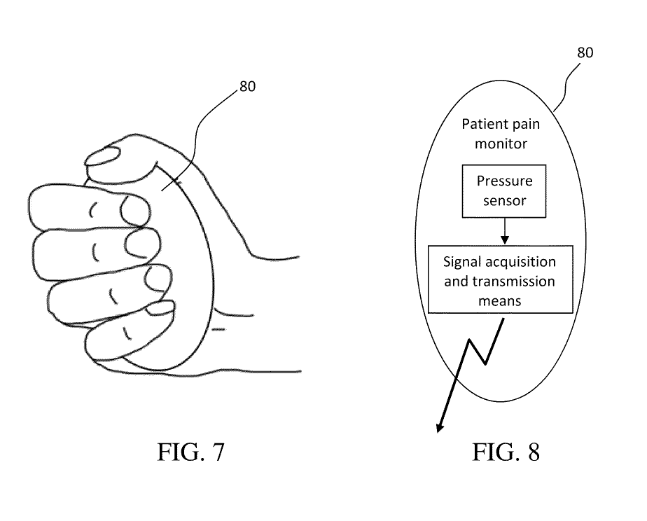

A third embodiment of the handheld pain monitor is shown in FIGS. 7 and 8. The housing in this case is inflated balloon 80 rather than a rigid shell shown for the first two embodiments. The patient experiencing pain squeezes the balloon as shown in 7. The squeezing force in this case is measured as balloon pressure. FIG. 8 shows the block diagram of this monitor. The electronic parts comprising the battery as a power source (not shown), the pressure sensor and the signal acquisition and transmission means can be installed inside as well as outside the balloon. The pressure sensor is adapted to measure the pressure in the balloon. Balloon pressure is then converted into a pain indicator signal and transmitted outside for further processing and recording.

Although the invention herein has been described with respect to particular embodiments, it is understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A handheld pain monitor comprising:
a housing sized and shaped to fit in a human hand, said housing adapted to be squeezed with a squeezing force representing a level of pain,
a sensor for measuring said squeezing force,
a data processor for converting said squeezing force into a signal representing a level of pain, and
a power source to provide electrical power to said sensor and said data processor.

2. The pain monitor as in claim 1, wherein said data processor is adapted for data transmission to an outside data receiver.

3. The pain monitor as in claim 1 further including a strap for attaching said monitor to a palm, whereby said monitor is retained in said human hand.

4. The pain monitor as in claim 1, wherein said housing comprises two rigid halves with said sensor placed between thereof.

5. The pain monitor as in claim 4, wherein said housing further including a pair of guiding pins fixedly attached to one half of said housing and slidingly attached to the other half, whereby one half of the housing is guided by said pins to slide towards the other when the housing is squeezed.

6. The pain monitor as in claim 5, wherein said halves are spaced apart with a gap therebetween and said guiding pins are spring-loaded, whereby reduction in said gap serves as a biofeedback indicating the degree of squeezing of said pain monitor.

7. The pain monitor as in claim 4, wherein said sensor is a force sensor and said data processor includes a load cell, an analog-to-digital converter, a microprocessor and a wireless data transmission module.

8. The pain monitor as in claim 1, wherein said housing is an inflated balloon, said sensor is a pressure sensor and said data processor is adapted to convert a pressure measured by said sensor into a signal representing a level of pain when said balloon is squeezed.

9. The pain monitor as in claim 8 further including a signal acquisition and transmission means with said data processor forming a part thereof.

10. The pain monitor as in claim 9, wherein said signal acquisition and transmission means being configured for transmitting said signal representing the level of pain to a data receiver for further processing.

11. The pain monitor as in claim 10, wherein said signal acquisition and transmission means further configured for wireless transmission of said signal representing the level of pain.

12. The pain monitor as in claim 10, wherein said signal acquisition and transmission means further configured for transmission via a cable of said signal representing the level of pain, said cable connecting said pain monitor to said data receiver.
